(19) United States
(12) Patent Application Publication
(10) Pub. No.: US 2024/0050914 A1
(43) Pub. Date: Feb. 15, 2024

(54) Method for Generating Solid Capsules

(71) Applicant: Microcaps AG, Schlieren (CH)

(72) Inventor: Kaj Pletscher, Zürich (CH)

(21) Appl. No.: 18/278,033

(22) PCT Filed: Feb. 21, 2022
(86) PCT No.: PCT/EP2022/054242

§ 371 Date: Aug. 21, 2023

(30) Foreign Application Priority Data
Feb. 26, 2021 (CH) 00212/21

(51) Int. Cl. B01J 13/04; B01J 13/20

(52) U.S. Cl. CPC B01J 13/043; B01J 13/206

(57) ABSTRACT

A method for generating capsules includes: a. providing in a first chamber a dispersed phase, the dispersed phase including a solution including a first solvent and a matrix-forming agent, the matrix-forming agent is a solid in its pure state and the first solvent and the matrix-forming agent are configured such that the matrix-forming agent is soluble in the first solvent; and b. providing in a second chamber a continuous phase, the continuous phase including a second solvent. The first and second chambers are fluidic connected by channel(s). The method further includes: c. guiding the dispersed phase from the first chamber through the channel(s) into the second chamber to form an emulsion or a dispersion including a plurality of droplets of the dispersed phase, in the continuous phase; and d. removing the first solvent from the droplets of the dispersed phase and solidifying the matrix-forming agent to form a capsule.

### CROSS-REFERENCE TO RELATED APPLICATIONS

This application is the United States national phase of International Application No. PCT/EP2022/054242 filed Feb. 21, 2022, and claims priority to Swiss Patent Application No. 00212/21 filed February 26, 2021, the disclosures of each of which are hereby incorporated by reference in their entireties.

### BACKGROUND OF THE INVENTION

### Field

The present disclosure relates to a method for generating capsules, particularly microcapsules and capsules generated by this method, as well as a device for generating such capsules.

### Description of Related Art

Capsules with particle sizes of 5.0 mm or less, particularly microcapsules with particle sizes of less than 1 mm, i.e. of between 0.1 μm and 0.5 mm, in particular between 1 μm and 0.5 mm, have found widespread application in the field of pharmaceutics, cosmetics, diagnostics, food and material science. Such capsules may be produced from an emulsion of monodisperse droplets in a continuous phase. Monodispersity increases stability, allows to accurately control volumes in multiple chemical or biological reactions, to increase the surface to volume ratio and enables the production of periodic structures, reproducible processes and ensures equal reaction conditions. Microfluidics offers an exquisite platform to precisely form monodisperse droplets. The monodisperse droplets can be cured for generating microcapsules for encapsulation of active ingredients such as drugs, fragrances, flavors, peptides, living material, such as bacteria or phages etc. fertilizers, pesticides, and other active substances for well-being.

Currently, most industrial processes for generation of microcapsules employ spray drying, high speed rotation with high shearing forces, ultrasonication, mixing and/or shaking. Noteworthy, such processes not only have a profound energy consumption, but generally also have the disadvantage of poor control over the capsule size, size distribution as well as surface properties of the capsules. However, these parameters are crucial for many applications. Furthermore, several compounds of interests and other components can be sensitive to the typical high shear forces and/or temperatures applied in prior art processes.

### SUMMARY

Hitherto known methods for producing capsules from monodisperse droplets show significant limitations. Known methods suffer from a severely limited overall operational capacity and/or from poor reproducibility and size control. However, controlling the size of the capsules is of utmost importance for various applications, particularly for applications in the pharmaceutical, fragrance and flavor industry. Furthermore, for many applications it is important to control the surface properties of the capsules, i.e. to ensure equal surface properties over each capsule and to provide even surfaces.

It is therefore a general object to advance the state of the art of generating capsules, particularly microcapsules, and preferably to overcome the disadvantages of the prior art fully or partially. In favorable embodiments, a method and a device for producing such capsules is provided allowing for accurate control of the capsule size and size distribution. In further advantageous embodiments, a method and a device for producing such capsules is provided allowing for accurate control of the surface properties of the capsules, particularly for allowing to produce capsules with equal surface properties over each capsule and/or even surfaces. In particular embodiments, capsules formed may have a maximum difference of 5%, in particular of 1% with respect to a perfect sphere. In further particular embodiments, the coefficient of variation (CV) regarding the size distribution is below 10%, preferably below 8% or even below 6%. In some favorable embodiments, the surface properties of the capsules vary only by a maximum of 10%, preferably by a maximum of 5%.

In a first aspect, the general object is achieved by a method for generating capsules, particularly microcapsules, the method comprising the steps:
a. Providing in a first chamber a dispersed phase, the dispersed phase being a solution comprising a first solvent and a matrix-forming agent, wherein the matrix-forming agent is a solid in its pure state and wherein the first solvent and the matrix-forming agent are configured such that the matrix-forming agent is soluble in the first solvent;
b. Providing in a second chamber a continuous phase, the continuous phase comprising a second solvent;
   wherein the first chamber and the second chamber are fluidic connected by one or more channels, particularly by micro-channels; the method further comprising the steps:
c. Guiding the dispersed phase from the first chamber through the one or more channels into the second chamber to form an emulsion or a dispersion comprising a plurality of droplets, particularly microdroplets, of the dispersed phase, in the continuous phase;
d. Removing the first solvent from the droplets of the dispersed phase and thereby solidifying the matrix-forming agent to form a capsule, preferably a microcapsule.

One advantage of the method according to the present disclosure is that it relates on the principles of step emulsification, i.e. guiding the dispersed phase of step a. through the micro-channels enables to accurately control the size and to ensure uniform size distribution of the emulsion or dispersion formed in step c. Subsequently in step d. the first solvent is removed, for example by evaporation, sublimation or extraction. This step may occur after removal of the emulsion or dispersion from the first chamber. Even though removal of the first solvent may cause a shrinkage of each individual capsule with respect to the corresponding droplet, the size distribution of the capsules remains constant. In any case, as outlined further below, the shrinkage can be calculated. Furthermore, the method allows for a much more rapid production of capsules than the methods known in the prior art. The method disclosed herein allows for a capsule production of 100 g/h or more, or even up to 500 g/h, particularly of up to 150 kg/h or even up to 200 kg/h.

It is understood that steps a. and b. must not necessarily be performed in this order. It may also be possible to first perform step b. and then step a. or perform them simultaneously. Typically, step d. is performed after step a., b. and c.

It is understood that the emulsion or dispersion formed in step c. comprises a plurality of monodisperse droplets, preferably with a particle size of 5.0 mm or less, particularly microdroplets, preferably with a particle size of 1 mm or less, comprising the dispersed phase of step a. within the continuous phase. The term “dispersed phase” in step a. does not mean that this phase in step a. cannot be monophasic, but means that it is this phase, which is to be dispersed in step c. The dispersed phase of step a. can be monophasic, but it may in some embodiments also be biphasic, i.e. be an emulsion.

It is understood that the matrix-forming agent is a solid in its pure state, i.e. it can be in the solid state at specific conditions, in particular at standard conditions (temperature of 273.15 K and a pressure of 1 atm.) or at room temperature (293.15 K) and a pressure of 1 atm. The pure state is a state in which the matrix-forming agent is for example not dissolved in a solvent or mixed with any other compound.

The matrix-forming agent and the first solvent are selected such that the matrix-forming agent can be at least partially or completely dissolved in the first solvent, either at standard conditions or at other conditions, for example at temperatures higher than 0° C. or higher than 20° C., or between 20° C. and 150° C. The matrix forming agent may have a solubility of at least 0.1 wt. %, in particular between 0.1 wt. % to 200 wt. %, preferably 5 wt. % to 100 wt. %.

Upon removal of the first solvent in step d., the matrix-forming agent having initially been completely dissolved in the first solvent precipitates and solidifies, forming a matrix. The capsules generated are typically solid capsules, in particular compact capsules. It is understood that the term “capsule” as used herein may in some embodiments also include beads, or other suitable structures.

Thus, the matrix-forming agent forms the matrix, i.e. the solid capsule, as a result of removing the first solvent. The matrix-forming agent does therefore typically not undergo a chemical reaction in the method according to the present disclosure.

In order to determine the size of each capsule produced with a method according to an embodiment of the present disclosure, the following equation can be used:

$$S = \frac{{D}_{d}}{{D}_{p}} = \sqrt[3]{\frac{{ρ}_{p}}{{X}_{p} \cdot {ρ}_{d}}}$$

wherein
S is the shrinkage factor;
D_{d} is the droplet diameter of the droplet in the emulsion or dispersion formed in step c.
Dₚ is the capsule diameter;
Pₚ is the density of the generated capsule
Xₚ is the mass fraction of the original droplet
Pₚ is the density of the droplet in the emulsion or dispersion formed in step c.

As used herein, the term “microcapsule” generally may refer to a capsule with a particle size of less than 4 mm, preferably between 0.1 μm and <4 mm, more preferably between 0.1 μm and <1 mm, more preferably between 1 μm and <1 mm, more preferably between 2 μm and <0.5 mm. Concomitantly, a microdroplet has a droplet size, i.e. a diameter of less than 4 mm, preferably between 0.1 μm and <4 mm, more preferably between 0.1 μm and <1 mm, more preferably between 1 μm and <1 mm, more preferably between 2 μm and <0.5 mm.

The second solvent of the continuous phase is typically different from the first solvent of the dispersed phase. Furthermore, the first solvent and the second solvent are typically immiscible, in particular at room temperature and a pressure of 1 atm. or at standard conditions.

The first solvent and/or the second solvent may be selected from dichloromethane, chloroform, acetonitrile, DMSO, water, ethyl acetate, dioxane, acetone, anisole, DMF, THF, Et₂O, alkyl alcohols, such as methanol, ethanol or propanol, as well as toluene, C₅₋₃₀ alkyls, such as pentane, hexane, and the like.

For example, in some embodiments, the dispersed phase is an aqueous phase and the first solvent is water. A suitable second solvent of the continuous phase may be dichloromethane or toluene. As an alternative example, the first solvent can be dichloromethane or toluene and the second solvent can be water. Alternatively, the dispersed phase may be an oil phase and the first solvent is pentane and a suitable second solvent may be acetonitrile or water.

The first chamber and second chamber are typically separated from each other with the exception of the one or more channels connecting the first chamber with the second chamber. A chamber as used herein is configured for being filled with a solution. Typically, the chambers are hermetically closed with the exception of inlets, channels and outlets. However, the chambers, in particular the second chamber, may also, at least indirectly or directly, be open to the environment.

The first chamber has typically a first fluid inlet for introducing, particularly continuously introducing, the dispersed phase in step a. into the first chamber and the second chamber has a second inlet for introducing, particularly continuously introducing, the continuous phase into the second chamber in step b. The second chamber also has an emulsion or dispersion outlet for removing, preferably continuously removing, the emulsion or dispersion formed during step c. from the second chamber.

The method according to the present disclosure is in general a step emulsification method.

It is understood that the one or more channels each comprise an inlet opening into the first chamber and an outlet opening into the second chamber. Thus, the one or more channels are directly connected to the first chamber and the second chamber. Typically, the first chamber and the second chamber are fluidic connected by multiple channels, i.e. at least 10, at least 20, at least 30, at least 50 or at least 100 channels. Preferably, the first chamber and the second chamber are fluidic connected by 1 to 30 000 000, preferably 20 to 1 000 000, more preferably 100 to 1 000 000 channels. Typically, the channels are arranged essentially parallel to each other.

For example, the one or more channels may have a diameter in the range of 0.25 μm to 2000 μm, preferably 2 μm to 1200 μm, in particular of 5 μm to 1100 μm. The channels are typically micro-channels. For example, each channel may have a cross-sectional area of 0.04 μm² to 4 000 000 μm², preferably 4 μm² to 640 000 μm².

In some embodiments channel length is in the range of 0.05 mm to 20 mm, particularly between 0.1 mm to 20 mm, particularly 0.1 mm to 5 mm, particularly 0.5 to 20 mm.

In further embodiments, the aspect ratio of each channel, which is defined as channel length/minimum diameter, is 5 to 1000, particularly, 10 to 500, more particularly 10 to 50. In some embodiments, the length of the channel may be in the range of 0.05 mm to 20 mm, particularly between 0.1 mm to 20 mm, particularly 0.1 mm to 5 mm, particularly 0.5 to 20 mm. It is understood that the channel length extends along the channels longitudinal axis, i.e. along the direction of flow in the operative state.

In certain embodiments each channel comprises a channel outlet with a cross-sectional area which is larger than the cross-sectional area of the remaining part of the respective channel. The channel outlet typically opens into the second chamber. In the longitudinal direction, i.e. in the direction of flow, the channel outlet has a typical length of several micrometers, for example 200 μm to 20 mm, preferably 500 μm to 5 mm. The channel outlet may for example be funnel shaped, V-shaped or U-shaped. In some embodiments, the channel outlet may have an elliptical contour. In particular, the channel outlet is not rotational symmetric, thus having a ratio of length/width of 3 and higher. Hence, the channel outlet may not have a circular or square shaped cross-section. Such a channel outlet enables the detachment of a droplet without external force. As a result, droplet formation of the dispersed phase in the continuous phase is decoupled and thus essentially independent from the flow rate. According to the Young-Laplace equation, the pressure at an immiscible liquid interface is higher at the channel outlets than in the second chamber. Thus a pressure gradient along the direction of the flow is generated, which causes the detachment of the fluid thread into individual droplets. Thus a pressure gradient is generated at the end of the channel, which facilitates the detachment of the fluids boundary layer and therefore the formation of the individual droplets. When reaching the channel outlet, the pressure gradient of the dispersed phase in and outside of the channel a droplet detaches without external force. Such a nozzle is advantageous, as it decouples the flow rates from the emulsification process.

Typically, each channel is defined by channel walls. The channel walls may be curved, i.e. the channel walls may be convexly or concavely shaped towards the channel outlet. Furthermore, each channel may comprise a constriction with a cross-section which is smaller than the cross-section of the rest of the channel and wherein the constriction is arranged adjacent the channel outlet. Thus, the constriction is arranged between the channel outlet and the rest of the channel.

In further embodiments, the cross-sectional area of each channel outlet is 0.12 to 36 000 000 μm², preferably 12 to 5 760 000 μm². In particular, in embodiments in which the channels are comprised in a membrane, total open area of the second side of the membrane may be 300% to 1500%, preferably 400% to 900%, larger than total open area of the channels at any other given position, such as the main section and/or the channel inlets. The channel inlets typically open into the first chamber.

In some embodiments, the one or more channels may be comprised in a membrane separating the first chamber from the second chamber. In such embodiments, the membrane can be flat, for example disc-shaped. The membrane typically has a first side facing the first chamber and a second side, being opposite to the first side and facing the second chamber. Thus, the first side of the membrane may partially limit and/or define the first chamber and the second side of the membrane may partially limit and/or define the second chamber. The one or more channels, typically multiple channels, extend from the first side to the second side through the membrane. Each channel comprises a channel inlet arranged at the first side, a channel outlet arranged at the second side and a main section being arranged between the channel inlet and channel outlet, wherein the channel outlet comprises a shape deviating from the shape of the main section.

In some embodiments the membrane thickness is in the range of 0.05 mm to 25 mm, particularly between 0.1 mm to 25 mm, particularly 0.5 to 25 mm particularly 0.1 mm to 5 mm. Typically, the thickness of the membrane may be equal to the total length of each channel.

The membrane may typically be a monolayer membrane. That is, the membrane is made from a single piece. Preferably, such a membrane is made from a massive material and does not contain any phase interfaces or transition areas in addition to the multiple channels of the membrane. Such a membrane is advantageous for the quality of the generated droplets, as any phase interfaces and transitions are detrimental to droplet formation and droplet stability.

In some embodiments, the membrane may be exchangeable. The multiple channels of the membrane are typically micro-channels. For example, each channel may have a cross-sectional area of 0.04 μm² to 4 000 000 μm², preferably 4 μm² to 640 000 μm².

In further embodiments, the channel outlet may be wedge-shaped. In particular, the channel outlet may comprise an elliptical cross-section with respect to a transversal plane being perpendicular to the extending channel, i.e. the channel outlet may be larger in a first direction than in a second direction.

In further embodiments, the second side of the membrane comprises a total open area that is larger than the total open area of the first side. Such a membrane has the advantage that high quality droplets are generated, even at flow rates of up to 5 L/h. In some embodiments, the flow rate per channel may be between 1 μL/h to 50 mL/h, preferably 10 μL/h to 5 mL/h.

In certain embodiments, each channel outlet may have an elliptical contour. Thus, the channel outlet may have an elliptical cross-section with respect to a plane being transversal to the extending channel and being parallel to the first or second side of the membrane. Channel outlets with an elliptical contour have a beneficial effect on the quality of the formed droplets, as any edges within the channel may lead to unstable and in homogeneous droplets.

In some embodiments, the membrane is disk-shaped. Such a membrane may have a circular contour. Alternatively, the membrane may have an angular, particularly a triangular or rectangular, contour.

In further embodiments, the membrane comprises 0.06 to 600 000 channels/cm², preferably 20 to 30 000 channels/cm².

In some embodiments, the membrane is made of glass or a polymeric material, such as polymethyl(meth)acrylate, polyolefin, such as polypropylene or polyethylene, or PTFE or of a metallic material, such as steel. The membrane may be covered with a coating, for example a hydrophobic or hydrophilic coating in order to avoid adhesion of the dispersed phase.

In some embodiments, the continuous phase comprises additionally at least one first surfactant. The droplets formed during step c. are stabilized by the at least one first surfactant and thus their size can remain essentially constant over a longer time, even after the droplets are removed from the second chamber.

In some embodiments, the at least one first surfactant is selected from polyvinyl alcohol (PVA), Tween® derivatives, such as Tween® 20 (polyoxyethylene (20) sorbitan monolaurate), Tween® 60 (polyethylene glycol sorbitan monostearate), Tween® 80 (polyoxyethylene (20) sorbitan monooleate), Tween® 85 (polyoxyethylenesorbitan trioleate), sodium dodecyl sulfate (SDS), polyglycerol polyricinoleate (PGPR), Span® derivatives, such as Span® 80 (sorbitane monooleate), Span® 85 (sorbitane trioleate) and Lubarfil® 1944 (triglycerides based on oleic and linoleic acid). Furthermore, the first surfactant may be a solid particle, depending on the application preferably a hydrophobic hydrophilic or Janus-type particle, configured for providing a pickering emulsion. For example, the solid particle may be colloidal silica.

In some embodiments, the amount of the at least one surfactant in the continuous phase is between 1 wt. % to 5 wt. %. The at least on second surfactant ensures stability of the formed emulsion or dispersion.

In some embodiments, the continuous phase consists of the second solvent and the at least one surfactant.

In some embodiments, the dispersed phase in step a. additionally comprises at least one first compound of interest. The compound of interest may be selected from a protein, small molecule, particularly a fragrant or flavor, cosmetic ingredient, active pharmaceutical ingredient such as cannabinoids, hemp extracts, caffeine, melatonin or hyaluronic acid; antibody, peptide, enzyme, RNA, DNA, vitamin and micro-organisms, including bacteria, viruses including phages, algae, cells, fungi, and bacteria supporting components, such as sugars, buffers, nutrients, reduction and oxidizing agents, etc. The at least one first compound of interest may for example be mixed with the first solvent in a suitable concentration. The at least one compound of interest may be dissolved or dispersed, i.e. being present as micro-particles with a particle size that is smaller, in particular 50% smaller, than the diameter of the at least one channel connecting the first chamber and the second chamber. In embodiments in which a compound of interest is employed, encapsulation efficiencies of 95% or higher (with respect to the encapsulation of the compound of interest) are observed with the method according to the present disclosure, which is significantly larger than with methods known in the prior art.

In specific embodiments, the at least one compound of interest is a living organism, in particular a microorganism, such as bacteria, a virus, including a phage, or a single cell. In some embodiments, the living organism may be provided in a dormant state into the dispersed phase. It is understood that the dormant state of a living organism relates to an inactive state.

The method according to the present disclosure is particular suitable for encapsulating living organisms, because the method exerts only marginal shear forces as compared to the method of the prior art. Furthermore, the encapsulation efficiency is significantly higher than of methods known in the prior art. It is possible to reach encapsulation efficiencies of up to 90% or even up to 95% with respect to the living organism.

In some embodiments, the method is performed at room temperature, which is highly beneficial for encapsulating living organisms, as the viability is increased.

Furthermore, by guiding the dispersed phase through the one or more channels, the channel dimension, in particular the channel diameter, dictates the amounts of living organism per droplet and thus the mount of organism per capsule formed. Therefore, by choosing predefined channel dimensions, accurate control of organism loading per capsule is possible.

In some embodiments, the channel diameter is chosen such that it is at least 6 times larger than the size of the living organism, or also any other solid particle.

In some embodiments in which the at least one compound of interest is a living organism, the living organism, such as cells or bacteria is provided by cultivation prior to being added into the dispersed phase. For example, cultivation may be performed on a suitable nutrient medium, such as agar-agar. In certain embodiments, viability of the living organism is monitored during cultivation and the living organism freeze dried when the viability reaches its maximum and subsequently added to the dispersed phase.

In certain embodiments it is beneficial to deoxygenate the water of the dispersed phase and/or the continuous phase. Deoxygenating can be achieved by common laboratory techniques, such as degassing with inert gases, such as argon or nitrogen, or by the freeze-pump-thaw technique. Such deoxygenating is beneficial, because the living organism can be maintained in its dormant state.

In some embodiments, the dispersed phase additionally comprises nutritional components for the living microorganism, such as sugars, electrolyte solutions, and the like.

In certain embodiments, the dispersed phase additionally comprises buffer solutions configured to maintain a pH suitable for the corresponding living organism.

The dispersed phase may also comprise one or more additional solvents, e.g. one or more co-solvents, being different from the first solvent but being miscible with the first solvent. In some embodiments, such one or more additional solvents may be immiscible or miscible with the second solvent of the continuous phase. In certain embodiments, the one or more additional solvent is different from the second solvent. In some embodiments the one or more additional solvent is selected such that the at least one compound of interest can be completely dissolved in the one or more additional solvent. Thus, the one or more additional solvent may facilitate to dissolve the at least one compound of interest in the dispersed phase. For example, if the first solvent is dichloromethane and the compound of interest is not sufficiently soluble in dichloromethane but sufficiently soluble in methanol, methanol can be used as an additional solvent, i.e. as a co-solvent, in the dispersed phase. In some embodiments, the dispersed phase may therefore comprise a solvent mixture of different solvents. In some embodiments, the relative amount in the dispersed phase of the one or more additional solvents, or all additional solvents, is lower than the amount of the first solvent. For example, the relative amount of the first solvent may be larger than 50 wt. %, in particular larger than 75 wt. %, in particular larger than 85 wt. %, in particular larger than 90 wt. %, in particular larger than 95 wt. % than the amount of the one or more additional solvents in the dispersed phase.

In some embodiments the total amount of the one or more additional solvents, e.g. one or more co-solvents, of the dispersed phase is up to 10 wt. %, in particular up to 5 wt. %. For example, the total amount of the one or more additional solvents, e.g. one or more co-solvents, of the dispersed phase is between 0.1 wt. % and 5 wt. %, particularly between 1.5 wt. % and 5 wt. %.

In some embodiments, step a. comprises dissolving the matrix-forming agent in the first solvent to provide the dispersed phase. Dissolving the matrix-forming agent may be achieved in a separate vessel or reactor and the dispersed phase is subsequently provided into the first chamber, which is different from the vessel or reactor.

In some embodiments, step d. comprises evaporating the first solvent from the droplets of the dispersed phase. Evaporation may be achieved by heating the formed emulsion or dispersion and/or by applying a negative pressure.

Alternatively, step d. may comprise sublimation of the first solvent. Such sublimation may for example comprise freezing of the formed emulsion or dispersion and subsequently applying a negative pressure.

In some embodiments, step d. comprises the extraction the first solvent from the droplets of the dispersed phase into the continuous phase. Such an extraction has the advantage that a particular equal size distribution of the capsules can be achieved. Furthermore, even surface characteristics of each capsule can be achieved, i.e. a coefficient of variation of less than 10% or even less than 9% or less than 8%, can be achieved. The capsules formed may have a maximum difference of 5%, in particular of 1%, with respect to a perfect sphere.

Extraction may for example be achieved selecting the first solvent and the second solvent such that first solvent has a solubility of 0.1 wt. % to 40 wt. %, particularly of 0.1 wt. % to 10 wt. %, particularly of 0.25 wt. % to 5 wt. %, at 25° C. and 1 atm. Thus, the first solvent and the second solvent are configured such that they can form an emulsion or dispersion, however as the first solvent is slightly miscible with the second solvent, the first solvent can be extracted from the droplets of the emulsion or dispersion thereby effecting precipitation, respectively solidification of the matrix forming agent to form a matrix and thus a solid capsule. A suitable solvent combination fulfilling these features may for example be dichloromethane as the first solvent and water as the second solvent.

Alternatively, extraction may be achieved by adding a third solvent to the emulsion or dispersion formed in step c. The third solvent may for example be added directly to the second chamber or after the emulsion or dispersion has been removed from the second chamber. The third solvent is typically different from the first solvent and/or the second solvent.

The third solvent may in some embodiments have a higher solubility for, respectively with, the first solvent of the dispersed phase as compared to the solubility of second solvent of the continuous phase for, respectively with, the first solvent, thereby increasing the extraction rate of the first solvent from the droplets formed.

If the extraction rate is too high, i.e. if the miscibility of the first solvent with the second solvent is sufficiently high, the third solvent may in some embodiments have a lower solubility for, respectively with, the first solvent of the dispersed phase as compared to the solubility of second solvent of the continuous phase for, respectively with, the first solvent, thereby decreasing the extraction rate. This may be beneficial, in order to control size distribution and overall surface characteristics of the capsules formed. Depending on the choice of the first solvent and the second solvent it may be beneficial to decrease the extraction rate in order to avoid that extraction commences already during step c and thus may lead to channel clogging.

In some embodiments, the extraction of step d. is performed in an extraction column. The extraction column is fluidic connected to the second chamber, i.e. to the dispersion outlet of the second chamber. The extraction column may also be an integral part of the second chamber. In certain embodiments, the extraction column may have an inlet configured for providing, preferably continuously providing, the third solvent.

Typically, the emulsion or dispersion formed in step c. and/or the capsules already formed flow continuously through the extraction column.

It is understood that the extraction column typically has at least one outlet for removing the capsules and/or droplets.

In some embodiments, the extraction column may have a length from 0.2 m to 50 m, in particular from 0.4 m to 20 m, in particular from 0.6 m to 5 m.

In some embodiments, the extraction column may have a volume of 40 mm³ to 40 m³.

In some embodiments, the extraction column may be tubular, in particular cylindrical, having preferably a diameter of 0.5 mm to 0.5 m, particularly from 10 mm to 0.25 m, particularly from 0.01 m to 0.25 m.

In some embodiments, the extraction column may be arranged such that after the emulsion or dispersion formed in step c. enters the extraction column, the emulsion or dispersion moves vertically upwards, i.e. in the opposite direction of the gravitational force vector, or downwards, i.e. in the direction of the gravitational force vector, towards an outlet of the extraction column.

In some embodiments, the dispersed phase in step a. is an emulsion of the first solvent and a fourth solvent, wherein the emulsion comprises at least one second surfactant. Is such embodiments, the first solvent and the fourth solvent are different from each other. The fourth solvent and the first solvent are typically immiscible with each other. Thus, in some embodiments, the dispersed phase may be an oil in water emulsion or a water in oil emulsion. The at least one second surfactant stabilizes the emulsion. Such embodiments are useful, if the first solvent has to be chosen such that the compound of interest is not soluble in the first solvent.

In some embodiments, the at least one second surfactant is a nonionic surfactant, such as polyvinylacohol (PVA). Other suitable second surfactants may be starch, sodium 0.5-2 octenyl succinate, Tween® derivatives, such as Tween® 20 (polyoxyethylene (20) sorbitan monolaurate), or Span® derivatives, such as Span® 80 (sorbitane monooleate) or Span 85® (sorbitane trioleate). Alternatively, anionic or cationic surfactants can be used. Other suitable surfactants may be polyglyceryl-3-Polyricinoleate, sorbitan isostearate and/or polyglyceryl-3-polyricinoleate capric triglyceride. Furthermore, the second surfactant may be a solid particle, depending on the application preferably a hydrophobic hydrophilic or Janus-type particle, configured for providing a pickering emulsion. For example, the solid particle may be colloidal silica.

In such embodiments, the dispersed phase which is to be guided into the second chamber is already an emulsion. Thus, by guiding it through the one or more channels, particularly microchannels, an oil in water in oil emulsion or dispersion, or a water in oil in water emulsion or dispersion may be formed. Amongst others, this has the advantage that not only hydrophilic compounds of interest can be dissolved and thus evenly distributed in the dispersed aqueous phase, but also hydrophobic compounds of interest.

It is understood that in embodiments in which the dispersed phase in step a. is already an emulsion of the fourth solvent in the first solvent, the majority of the dispersed phase is composed of the first solvent. Typically, more than 60 wt %, particularly more than 70 wt %, particularly more than 80 wt %, particularly more than 90 wt %, particularly more than 95 wt %, particularly more than 99 wt % of the dispersed phase constitutes the first solvent.

The emulsion of the fourth solvent in the first solvent and/or the emulsion or dispersion formed in step c. is typically a stable emulsion. Thus, it may be stable for 60 min to 1 month, particularly 60 min to 24 h, particularly 60 min to 600 min, preferably from 100 min to 500 min. Such a stability ensures that the droplets are not directly destroyed, particularly during step c. and/or d.

In some embodiments, the matrix-forming agent is a polymer, in particular a pharmaceutically acceptable polymer. Examples for such polymers are poly lactic acid, poly(lactic-co-glycolic acid), polycaprolactone, polyglycolide, poly(methyl methacrylate), polyanhydrides, polyorthoesters, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid) or the like.

In some embodiments, prior to step d. the emulsion or dispersion formed in step c. is removed, preferably continuously removed, from the second chamber, thereby increasing the overall production rate.

In some embodiments, step d. is at least partially performed under continuous flow of the emulsion or dispersion formed in step c. Thus, the emulsion or dispersion formed in step c. is not only continuously removed from the second chamber, but step d. is also performed in continuous flow, which is particularly suitable if an extraction column is employed. This significantly increases the overall production rate and thus the process efficiency. Furthermore, equal reaction conditions are provided for all capsules thereby providing uniform capsules.

In further embodiments, a pressure of 1.01 bar to 2.0 bar, in particular 1.01 bar to 1.15 bar, preferably of 1.03 bar to 1.07 bar is applied to the first chamber, particularly during step c., and/or a pressure of 1.02 bar to 1.2 bar, preferably of 1.05 bar to 1.1 bar is applied to the second chamber particularly during step c. It is understood that these pressure values relate to absolute pressures, i.e. a pressure of 1.01 bar is a pressure which constitutes an overpressure of 0.01 bar with respect to the atmospheric pressure.

In some embodiments, the pressure applied to the first chamber and/or the second chamber can be provided by a pump system, such as, gear pumps, peristaltic pumps or levitating pumps.

In some embodiments, the method is performed in an open system device, i.e. a device which is not closed with respect to the environment. Such embodiments simplify accurate pressure control as uncontrolled flowrates can be avoided.

In some embodiments, the pressure applied to the first chamber is smaller than the pressure applied to the second chamber. It is understood that the first pressure can be adjusted by the pressure with which the dispersed phase is provided via the first fluid inlet of the first chamber to the first chamber and/or the second pressure can be adjusted by the pressure with which the continuous phase of step b. is provided via the second fluid inlet of the second chamber to the second chamber.

In some embodiments, step d. is performed in total for 0.01 min to 48 h min, particularly from 0.1 min to 12 h min, particularly for 0.2 min to 10 min. Thus after this time, capsule formation is complete.

In some embodiments, the formed capsules are isolated, dried, cured and/or preserved after step d. Isolation of the capsules can for example comprise filtering or sieving in order to separate the capsules from residual solvent, such as oil, and optionally washing of the capsules with water optionally including a tenside, such as sodium laurylsulfate (SDS), a Tween derivative, such as Tween® 20 (polyoxyethylene (20) sorbitan monolaurate, CAS 9005-64-5) or 80 (polyoxyethylene (20) sorbitan monooleate, CAS 9005-64-6), or PVA. Curing may for example comprise drying of the capsules, for example by an air stream or by freeze drying, in order to evaporate all or at least the majority of the unbound water.

According to a second aspect of the present disclosure, the general objective is achieved by an assembly of capsules, particularly microcapsules, comprising a plurality of capsules produced according to the method according to any of the embodiments described herein, in particular according to any embodiments of the first aspect of the present disclosure.

Each capsule is typically a solid capsule, in particular a compact capsule.

In some embodiments, the capsules of the assembly, in particular all capsules of the assembly, have an equal size distribution with a coefficient of variation of 10% or less, particularly of 8% or less, particularly of 6% or less, particularly of 5% or less, particularly of 4% or less.

The skilled person understands that the coefficient of variation may be calculated by the ratio of the standard deviation σ to the mean μ, i.e. the average capsule size of the capsules of the assembly.

In some embodiments, the assembly of capsules comprises more than 50 capsules, particularly more than 100 capsules, particularly more than 500 capsules, particularly more than 1000 capsules, particularly more than 10 000 capsules, produced according to the method according to any of the embodiments described herein.

In some embodiments, each capsule of the assembly of capsules has a particle size of less than 4 mm, preferably between 0.1 μm and <4 mm, more preferably between 0.1 μm and <1 mm, more preferably between 1 μm and <1 mm, more preferably between 2 μm and <0.5 mm.

In some embodiments, the capsules of the assembly, in particular all capsules of the assembly, have a maximum difference of 1% with respect to a perfect sphere. In particular, the surface of the capsules has a maximum difference of 5% or even of maximum 1% with respect to a perfect sphere.

According to a third aspect of the present disclosure, the general objective is achieved by device for generating capsules, particularly microcapsules, comprising a first inlet for supplying a dispersed phase, opening into a first chamber; a second inlet for supplying a continuous phase, opening into a second chamber, wherein the first chamber and the second chamber are fluidic connected by one or more channels, particularly by micro-channels, wherein each channel comprises a channel inlet opening into the first chamber and a channel outlet opening into the second chamber; a dispersion outlet for collecting the emulsion or dispersion of the dispersed phase in the continuous phase; an extraction column being fluidic connected to the dispersion outlet; wherein the first chamber is configured such that a flow rate of the dispersed phase through each individual channel is essentially uniform.

In the state of the art, an in homogeneous pressure distribution, in particular of the dispersed phase, enables only a small percentage of the channels to actively produce droplets. An equal pressure distribution over the first side however, allows for a steady flow of the dispersed phase into continuous phase and for the generation of droplets with a reproducible quality with a high throughput of up to 5 liters per hour, or even up to 100 liters per hour.

In certain embodiments, the second chamber may be made from glass or a transparent polymer, such as PTFE, polymethyl(meth)acrylate or polyoxymethylene, or from metals such as steel, aluminum or titanium. In general, the device may comprise a container, such as a glass container, which partially forms the second chamber. Together with the membrane, the container may form the second chamber. In some embodiments, the first chamber may be made from metal, for example aluminum or steel or from a transparent polymer, such as PTFE, polymethyl(meth)acrylate or polyoxymethylene.

The dispersion outlet may for example be in fluidic communication with the extraction column as described above. In some embodiments, the extraction column may be an integral part of the second chamber. The extraction column may at least partially be straight, i.e. non-curved. The straight part is typically directly connected to the dispersion outlet.

In some embodiments, the device comprises a reservoir for the third solvent, as described in any of the embodiments of the first aspect of the present disclosure. The reservoir is in fluidic communication with the extraction column via an extraction column inlet.

In some embodiments, the extraction column, in particular an extraction column outlet of the extraction column, is further in fluidic communication with a collection vessel for collecting the formed capsules.

In some embodiments, the dispersion outlet of the second chamber or the second chamber itself is arranged such that the emulsion or dispersion formed in step c. flows in the direction of the gravitational force vector upon being removed from the second chamber via the dispersion outlet. This is beneficial, because the free falling emulsion or dispersion ensures structural integrity of each capsule.

Furthermore, in some embodiments, any edges or changes of direction of the extraction column are essentially avoided between the dispersion outlet in direction of flow for about 1 cm to 30 cm, particularly between 1 cm to 10 cm. This ensures structural integrity of the capsules, before they aggregate or sediment.

In some embodiments, the first chamber is configured such that in an operative state, the pressure along the first side of the membrane is essentially isobaric. For example, the first inlet may comprise a nozzle for providing an isobaric pressure distribution over the first side of the membrane. In particular, a spray nozzle may be used. Alternatively, the first chamber may be shaped such that an isobaric pressure distribution over the first side of the membrane, respectively over all inlets of the channels, is provided.

In further embodiments, the first chamber has a rounded cross-section with respect to a cross-sectional plane, which is perpendicular to the membrane and rotationally symmetric with respect to a central longitudinal axis. The term “rounded cross-section” as used herein refers to a continuous curve without increments, particularly to a curve which has in the cross-sectional plane being perpendicular to the membrane, a radius of at least 1 mm, particularly at least 5 mm, particularly at least 10 mm. It is understood that the curvature in the cross-sectional view can be described as a part of a circle, in particular the osculating circle, with said radius. Thus, the sidewalls of the first chamber may continuously converge towards each other in the upstream direction. The central longitudinal axis is an axis extending in the longitudinal direction of the device, which is arranged in the center of the device and/or to an axis being perpendicular to the membrane and intersecting the center of the membrane. For example, the first chamber may have a U-shaped cross-section or may be concavely rounded or semi-circular. The rounded cross-section is typically edgeless and thus excludes edges, which would lead to an uneven pressure distribution when the dispersed phase is forced through the membrane. The shape of the first chamber may in general preferably be essentially rotationally symmetric to the central longitudinal axis.

In some embodiments the first chamber has the shape of a spherical dome or a truncated cone. Such shapes have the advantage that the material flow of the dispersed phase is equally distributed over the channel inlets opening into the first chamber, thereby helping to provide an equal pressure distribution adjacent to individual channel.

In certain embodiments, the first chamber has a hemispherical shape.

In some embodiments, the first inlet is arranged adjacent to a pole of the hemisphere-shaped or spherical dome first chamber, thereby providing for an equal pressure distribution over the channel inlets opening into the first chamber.

In certain embodiments, the dispersion outlet may essentially be arranged on the central longitudinal axis and/or the axis being perpendicular to the membrane and intersecting the center of the membrane. Preferably, the second chamber is tapered towards the dispersion outlet. For example, at least parts of the second chamber may be arch- or cone-shaped towards the dispersion outlet. These embodiments ensure that no droplets are entrapped and all are directly collectable via the dispersion outlet.

In some embodiments, the one or more channels are comprised in a membrane separating the first chamber and the second chamber comprising a first side facing the first chamber and a second side facing the second chamber, wherein the one or more channels extend from the first side to the second side of the membrane providing a fluidic connection between the first chamber and the second chamber, wherein each channel inlet is arranged on the first side of the membrane and each channel outlet is arranged on the second side of the membrane.

In some embodiments, the first inlet is arranged in an angle of essentially 90° or less with respect to the longitudinal axis of the one or more channels. This has the beneficial effect that the dispersed phase is not directly forced towards the channel inlets, thereby further enabling to provide a uniform pressure distribution over each channel of the membrane. For example, the angle between the first inlet and the longitudinal axis of the channels may be between 60° and 90°, particularly 75° and 90°. Preferably, the first inlet is essentially transversely, preferably perpendicularly, arranged to the multiple channels. Thus, in embodiments in which the channels are comprises in a membrane, the first inlet may be parallel to the first side of the membrane.

In certain embodiments in which the device comprises more than one channel, all channels are arranged essentially in parallel to each other.

In further embodiments the device comprises a membrane holder for mounting the membrane.

In certain embodiments, the device comprises a container holder for holding the container, which partially forms the second chamber. The container holder may be releasably secured to the membrane holder. The container holder and/or the membrane holder and/or the basis may be made from any suitable material preferably a plastic material, such as PTFE, polymethyl(meth)acrylate or polyoxymethylene or a metal, preferably steel.

Preferably, if the container is a glass container, a damping pad may be arranged between the glass container and the container holder for avoiding damaging and sealing the glass container.

In some embodiments the membrane holder comprises clamping means for mounting the membrane, the membrane holder and/or the clamping means being configured to accommodate membranes having various thicknesses. Typically, the clamping means may be adjustable. Examples for clamping means include screws, clamps, bolts, locks, etc.

In some embodiments, the device comprises a base, and preferably the first chamber is partially formed by the base.

In further embodiments the base and/or the membrane holder comprises at least one sealing to seal the membrane against the base and/or against the membrane holder. The sealing ring may be configured such that it circumferentially fully surrounds the periphery of the membrane. The sealing ring may also comprise a gas outlet in fluidic communication with the first chamber and being configured to vent any gas present in the first chamber out of the first chamber.

In some embodiments, the base and/or the membrane holder comprises a spacer ring. Such a spacer ring allows for employing differently thick membranes.

In some embodiments the first chamber comprises a gas outlet, particularly a fluidic switch such as e.g. a valve. The gas outlet and optionally the membrane, are arranged such that gas within the first chamber is during supplying the dispersed phase to the first chamber, in particular during the first/initial filling of the first chamber with the dispersed phase, directed towards the gas outlet and removed from the first chamber via the gas outlet.

In some examples, the membrane may be inclined with respect to the central longitudinal axis of the device. Thus, the angle in a cross sectional view along the central longitudinal axis between the central longitudinal axis and the first and/or second side of the membrane is different from 90°. For example, the acute angle between the second side of the membrane and the central longitudinal axis may be between 45° and 89°, preferably between 70° and 88°, more preferably between 78° and 87°. In such embodiments, the gas outlet may be arranged at the top edge of the first chamber, which is formed by the membrane and another chamber wall. This ensure that any residual gas, in particular air, being present in the first chamber, for example prior to using the device, rises to the membrane and due to the inclined arrangement of the membrane is directed to the top edge and thus to the gas outlet. Normally, the channels of the membrane are too narrow for air to pass through and therefore a gas outlet as described in the embodiments above enables to remove all remaining gas, which otherwise can negatively influence uniform droplet size and distribution or block the first fluid from reaching all the micro-channels, hence decreasing the throughput.

Typically, the gas outlet may be in fluid communication with the environment of the device.

In some embodiments the device comprises at least one heater to heat the dispersed phase and/or the continuous phase and/or at least one cooler to cool the dispersed phase and/or the continuous phase. It may be beneficial to heat or cool either of the phases, as curing of the generated dispersed droplets may be readily effected by a temperature changes, for example by allowing the emulsion or dispersion to cool. Typically, the at least one heater may provide enough thermal energy to heat the dispersed phase and/or the continuous phase up to 100° C., up to 125° C., or up to 150° C. The heater may for example comprise a heating bath, such as a water bath or an oil bath. Alternatively, the heater may be an IR-radiator, a heating coil, or any other suitable heater.

In further embodiments the device comprises a first reservoir for the dispersed phase and/or a second reservoir for the continuous phase. Both the first and second reservoir may be pressurized. For example, the reservoirs may be fluidic connected to a pressure source, such as a compressor. Alternatively, the reservoirs may be syringes and pressurized by a common syringe pump and/or a plunger or a peristaltic pump, gear pump or any other pumping system.

In some embodiments, a flow restrictor is arranged between the second reservoir for the continuous phase and the second chamber. Such a restrictor is beneficial, as the second chamber typically does not provide a significant flow resistance for the continuous phase. Thus, by using a flow restrictor, the device is more stable, as unintentional pressure differences, for example by fluctuating air pressure, can be avoided.

In further embodiments, the second inlet comprises a supply channel being at least partially circumferentially arranged around the central longitudinal axis, respectively the axis being perpendicular to the first and second side of the membrane and intersecting the center of the membrane. The supply channel comprises one or more openings into the second chamber. At least partially circumferentially arranged around the above mentioned axis means that the supply channel may have the contour of, a partial circle, such as a semi-circle or a third of a circle, etc. Preferably, the supply channel is fully circumferentially arranged around the central longitudinal axis, respectively the axis being perpendicular to the membrane and intersecting the center of the membrane. In such embodiments, the supply channel forms a ring-like structure. Preferably, the supply channel comprises multiple openings into the second chamber, which in particular are essentially uniformly distributed along the circumference of the supply channel. Typically, the one or more openings of the supply channel may be arranged in the direction of the dispersion outlet, i.e. such that the openings are facing the dispersion outlet. Embodiments comprising a supply channel have the advantage that the continuous phase can be uniformly and smoothly introduced into the second chamber without causing detrimental turbulences which negatively influence the uniform shape and size distribution of the generated microdroplets. In some embodiments, the one or more openings of the supply channel are arranged such that a vortex is generated when the continuous phase is provided into the second chamber. Particularly, the one or more openings may be tubular and the longitudinal axis of each tubular opening can be inclined with respect to the central longitudinal axis of the device. Typically, all tubular openings are uniformly inclined. The generation of a vortex is beneficial as firstly, a surface stabilizer which may generally be comprised in the first and/or the continuous phase may be more evenly distributed, which will thus enhance the stability of the formed emulsion or dispersion and secondly, because the transport of the generated emulsion or dispersion towards the dispersion outlet is accelerated, which is particularly beneficial if the density of the first and continuous phase is essentially equal.

Typically, the supply channel is arranged at the bottom of the second chamber, i.e. adjacent to the membrane. The supply channel may for example also be arranged circumferentially around the membrane. The supply channel may have a diameter of 2 mm to 100 mm, preferably 5 mm to 20 mm.

Alternatively, the second inlet may constitute a single inlet opening directly into the second chamber, preferably from a lateral side of the second chamber.

### BRIEF DESCRIPTION OF THE DRAWINGS

The terms Fig., Figs., Figure, and Figures are used interchangeably in the specification to refer to the corresponding figures in the drawings.

The herein described invention will be more fully understood from the detailed description given herein below and the accompanying drawings which should not be considered limiting to the invention described in the appended claims. The drawings are showing:

FIG. 1 a schematic representation of the method according to one embodiment of the present disclosure;

FIGS. 2a and 2b a schematic representation of the method according to another embodiment the present disclosure;

FIG. 3 view of a device for generating capsules, particularly microcapsules, according to a first embodiment of the present disclosure;

FIG. 4 a cross-sectional view of the device shown in FIG. 3;

FIG. 5 an exploded partially cut-out view of the device shown in FIG. 3;

FIG. 6 a schematic view of a device 1 according to another embodiment of the present disclosure;

FIG. 7 a schematic enlarged view of a second side of a membrane according to an embodiment of the present disclosure;

FIG. 8 a partial cross-sectional of a device according to another embodiment of the present disclosure;

FIG. 9 a cross-sectional of a device according to another embodiment of the present disclosure;

FIG. 10 shows another device which can be used in the method according to an embodiment of the present disclosure;

FIG. 11 shows the particle size distribution of an assembly of capsules according to an embodiment of the present disclosure comprising a plurality of capsules.

### DESCRIPTION

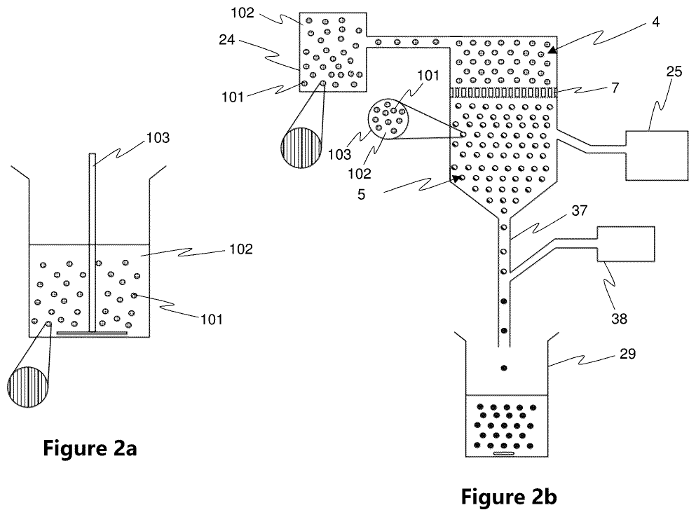

FIG. 2 illustrates schematically the method according to an embodiment of the present disclosure. The dispersed phase is provided in first chamber 4 from first reservoir 24 containing the dispersed phase and being in fluidic connection with first chamber 4. A continuous phase is supplied from second reservoir 25 to the second chamber 5. As can be seen, reservoir 25 is in fluidic communication with second chamber 5. First chamber 4 and second chamber 5 are separated from each other by membrane 7 having a first side 8 facing the first chamber and an opposing second side 9 facing the second chamber 5. Fluidic communication between first chamber 4 and second chamber 5 is established by channels 10 of membrane 7. The dispersed phase comprising a first solvent and a matrix-forming agent, is guided from first chamber 4 through channels 10 of membrane 7 into second chamber 5 containing the continuous phase, which comprises a second solvent and optionally at least one first surfactant, upon which microdroplets form, which represent an emulsion or dispersion of the dispersed phase in the continuous phase. This emulsion or dispersion is then continuously removed from second chamber 5 via dispersion outlet 6. Dispersion outlet 6 is directly connected to extraction column 37 which is configured such that the emulsion or dispersion exits the second chamber in the direction of the gravitational force vector and is arranged such that a straight flow path is provided, at least for a certain defined length. Extraction column 37 is in fluidic communication via a extraction column inlet with third reservoir 38 for a third solvent being configured to induce extraction of the first solvent from the formed droplets. Upon extraction of the first solvent, precipitation, respectively solidification, occurs and thus produces solid capsules. The capsules are then collected in collection vessel 29 being in fluidic communication with extraction column 37. Vessel 29, may be equipped with a stirrer.

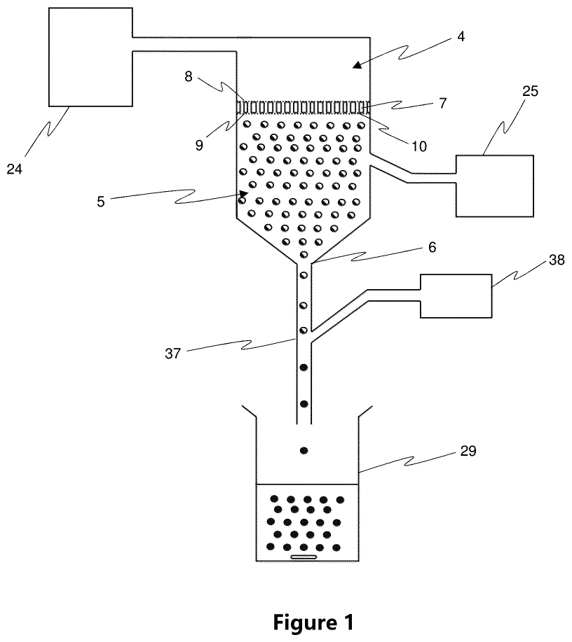

FIGS. 2a and 2b show schematically the method according to another embodiment of the present disclosure. It is understood that the reference signs being identical to the ones in FIG. 1 correspond to the same features unless stated otherwise. FIG. 2a shows the generation of a dispersed phase. In this embodiment, the dispersed phase in step a. is an emulsion of fourth solvent 101 in a first solvent 102, wherein the emulsion comprises at least one second surfactant. Formation of such an emulsion can be performed by mixer 103. The at least one second surfactant stabilizes the formed emulsion. This emulsion is then provided in FIG. 2b into first reservoir 24, from which it is provided into first chamber 4. The emulsion in first chamber 4 is then guided through channels 10. As the emulsion generally comprises as the major component first solvent 102, a step emulsification takes place as the emulsion reaches the channel outlet opening into second chamber 5, thereby forming a emulsion or dispersion of the dispersed phase, i.e. monodisperse droplets 103 in the continuous phase. It should be noted that the sizes of the droplets are exaggerated for clarity purposes. Furthermore, the relative size of droplets 101 with respect to droplets 103 does not resemble the reality. Each monodisperse droplet 103 in second chamber 5 now comprises one or more droplets 101 of the fourth solvent being dispersed in first solvent 102, as it illustrated in the enlarged view of a droplet. Thus the emulsion or dispersion in second chamber 5 may be considered as a “oil in water in oil emulsion” or as a “water in oil in water” emulsion. The formed emulsion or dispersion is removed from second chamber 5 via extraction column 37. Capsule formation may be effected as in the embodiment of FIG. 1.

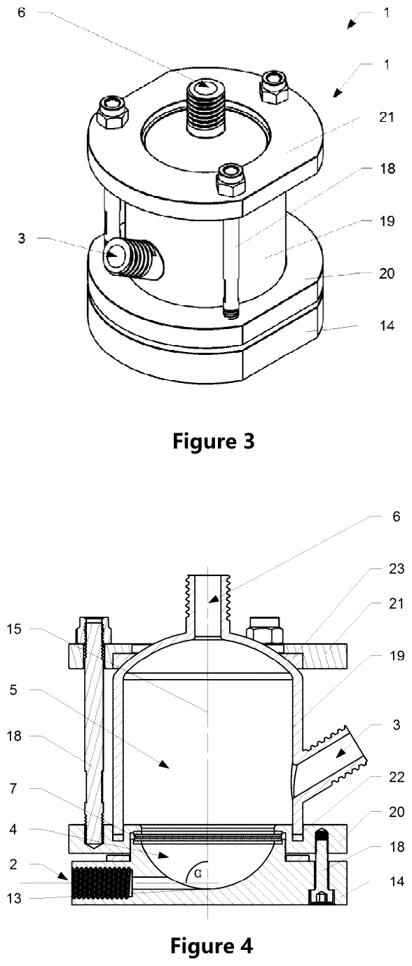

FIG. 3 depicts device 1 which can be used in a method according to the present disclosure for generating capsules. Device 1 comprises a container 19, which is made from glass and base 14 being made from metal. Base 14 comprises a first inlet (not shown, see FIGS. 2a and 2b) for supplying a dispersed phase, opening into a first chamber. The first chamber may be partly formed by base 14 and membrane 7 (see FIG. 3). Container 19 comprises second inlet 3 for supplying a continuous phase, opening into a second chamber and dispersion outlet 6 for collecting the emulsion or dispersion generated within the second chamber. The second chamber is being formed by container 19 and membrane 7 (see FIG. 3). Device 1 further comprises membrane holding structure 20 being fixedly connected to base 14. Furthermore, the device contains container holding structure 21, which is fixedly connected via clamping means 18 to membrane holding structure 20. As a result, container 19 is fixedly connected to base 14.

FIG. 4 shows a cross-sectional view of device 1 of FIG. 3. Device 1 comprises base 14 with first inlet 2 for supplying the dispersed phase. Inlet 2 opens into first chamber 4, which is partially formed by base 14. Device 1 further contains container 19 with second inlet 3 for supplying the continuous phase and dispersion outlet 6 for collecting the emulsion or dispersion of the dispersed phase in the continuous phase. Second inlet 3 opens into second chamber 5, which is partially formed by container 19. The first chamber and the second chamber are being separated by membrane 7. As can be readily seen from FIG. 4, the first chamber has a rounded cross-section with respect to the corresponding cross-sectional plane along the central longitudinal axis 15 and being perpendicular to membrane 7. In the particular embodiment shown, first chamber 4 has a semi-circular cross-section and may thus have the shape of a hemisphere. First inlet 2 is arranged in the region of pole 13 of the hemisphere. Second chamber 5 is tapered towards dispersion outlet 6, which is arranged on longitudinal axis 15 extending along the longitudinal direction of the device, intersecting the center of the first and second chamber, being perpendicular to membrane 7 and intersecting the center of the membrane. As can be seen, longitudinal axis 15 constitutes a central axis of the device in the longitudinal direction. In the embodiment shown, the second chamber is arch-shaped towards dispersion outlet 6. Thus, second chamber 6 has a U-shaped cross-section. First inlet 2 is arranged in an angle α of essentially 90° with respect to central axis 15 and the channels of the membrane, which are in general parallel to axis 15. Device 1 comprises membrane holder 20 and container holder 21, which are fixedly connected with each other via releasable clamping means 18. Membrane 7 is mounted to membrane holder 20 by clamping the membrane between membrane holder 7 and base 14. Membrane holder 20 is fixedly connected to base 14 via clamping means 18. For safely securing glass container 19 between membrane holder 20 and container holder 21, pad 23, which in the particular case is a foam pad, can be arranged between container 19 and container holder 21. Membrane holder 20 comprises groove 22, for receiving container 19.

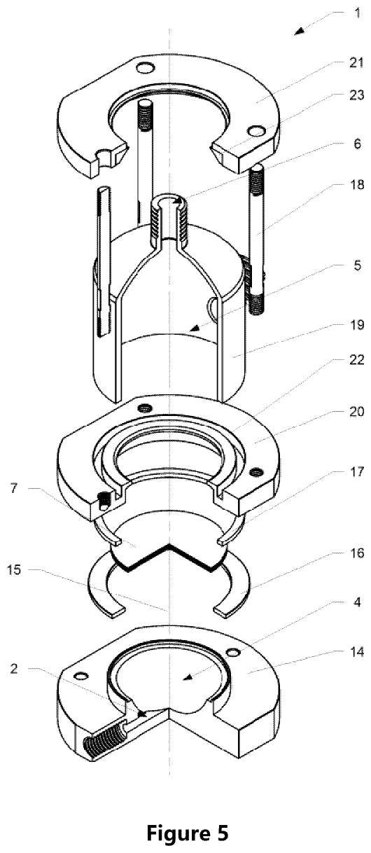

FIG. 5 shows an exploded view of partially cut device 1 of FIGS. 2a and 2b. As can be seen, the first chamber is partially formed by base 14 and has the shape of a hemisphere. First inlet 2, which is arranged in an angle of essentially 90° to central axis 15, is arranged on the pole of the hemisphere. Base 14 comprises spacer ring 16 which enables the use of different membranes with different thicknesses and membrane holder 20 comprises sealing ring 17. Membrane 7 is arranged between rings 16 and 17. The design of device 1 with adjustable clamping means 18 allows to employ membranes of various thicknesses. Membrane holder 20 further comprises circumferential groove 22 for receiving the lower end portion of container 19. Clamping means 18 fixedly and releasably connect membrane holder 20 with container holder 21.

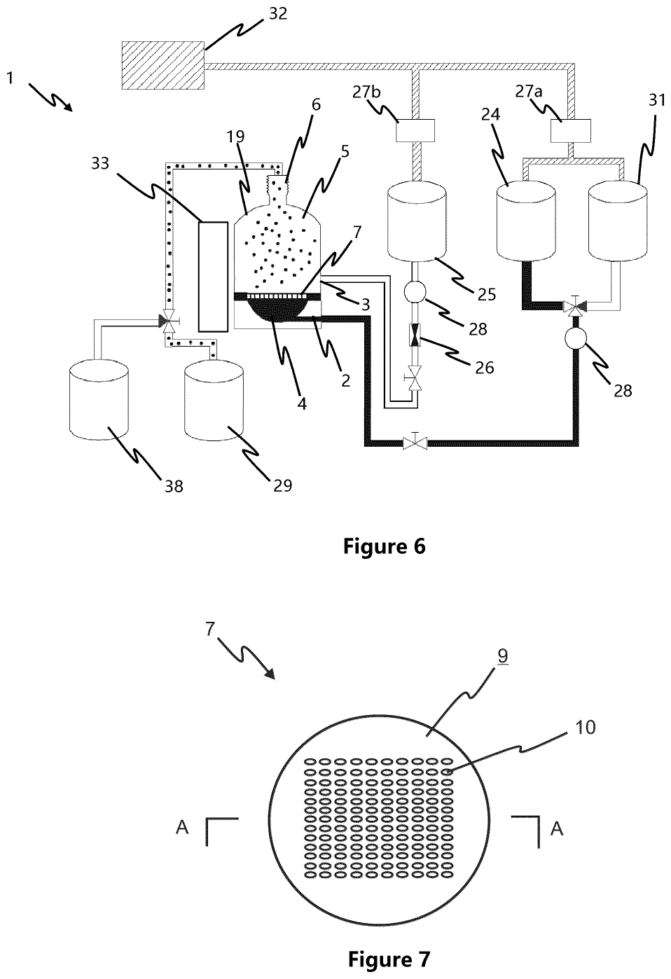

FIG. 6 shows a schematic view of a device 1 which may be used according to a preferred embodiment of the present disclosure. Second chamber 5 is formed by container 19 and membrane 7 which separates first chamber 4 from second chamber 5. Container 19 comprises dispersion outlet 6, which is in fluid connection with collection vessel 29. In general, the fluid flow may be controlled by a valve, such as a three-way valve. Device 1 further comprises first reservoir 24 which is in fluid communication with first chamber 4 which may either only server as a reservoir for providing the dispersed phase into first chamber 4 via first inlet 2 or which can also serve as the mixing vessel for preparing the dispersed phase. Arranged between first reservoir 24 and first inlet 2 is a flow meter 28 for measuring the fluid flow of the dispersed phase. First reservoir 24 is in fluid connection with pressure source 32. Furthermore, pressure regulator 27a is arranged between first reservoir 24 and pressure source 32. In addition to first reservoir 24, device 1 comprises rinsing reservoir 31 which is also in fluid communication with both first chamber 4 and pressure source 32. Rinsing reservoir 31 is configured for providing a rinsing solution into first chamber 4 for cleaning device 1 after its intended use. Device 1 further comprises heater 33 configured for heating the first and second chamber during the production of a dispersed phase. Furthermore, second chamber 5 is in fluid communication with second reservoir 25 for supplying second chamber 5 with the continuous phase. Flow restrictor 26 and flow meter 28 are arranged between second chamber 5 and second reservoir 25. In the embodiment shown, flow restrictor 26 is arranged behind flow meter 28 in the direction of flow. Second reservoir 25 is further in fluidic connection with pressure source 32. Additionally, a second pressure regulator 27b is arranged between second reservoir 25 and pressure regulator 27a. The device further comprises third reservoir 38 for a third solvent as described herein.

FIG. 7 shows a monolayer membrane 7 for generating a emulsion or dispersion of the dispersed phase in the continuous phase, which can be used in a method and/or a device as described in any of the embodiments disclosed herein. Membrane 7 has a first side 8 (not shown) and second side 9, which in an operative state faces a second chamber. Multiple micro-channels 10 extend through membrane 7. Each channel 10 has an elliptical contour. In addition, membrane 7 comprises membrane sealing ring 44, which circumferentially fully surrounds the periphery of the membrane.

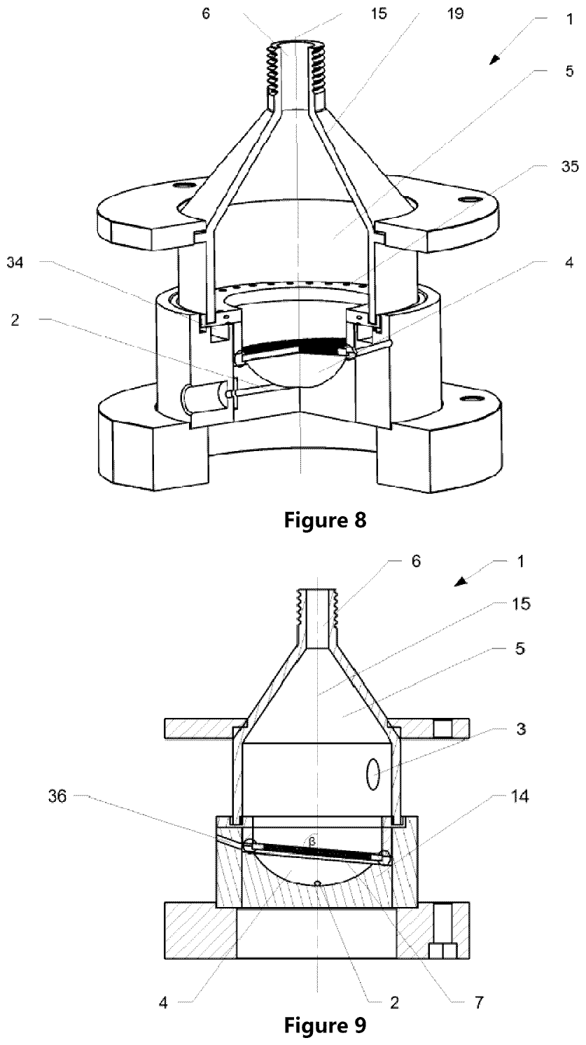

FIG. 8 shows a partial cross-sectional view of a device which can be used in embodiment of the present disclosure. The device 1 has a first inlet 2 for supplying a dispersed phase, which opens into first chamber 4 having a rounded cross-section. In the embodiment shown, first chamber 4 has the shape of a spherical dome with a radius at the base of the dome being smaller than the radius of the corresponding hypothetical full sphere. Second chamber 5 is at least partially defined by container 19. The device further comprises dispersion outlet 6 for collecting the generated emulsion or dispersion of the dispersed phase in the continuous phase. The corresponding membrane is not shown for better visualization. The second inlet opening towards the second chamber 5 comprises in the depicted embodiment a supply channel 34 being circumferentially arranged around central longitudinal axis 15 and/or the axis being perpendicular to the first and second side of the membrane and intersecting the center of the membrane. The supply channel 34 comprises a plurality of openings 35 into second chamber 5. Openings 35 are uniformly distributed along the circumference of the supply channel and are arranged in the direction of dispersion outlet 6. In the embodiment shown, supply channel 34 forms a ring-like structure, being arranged at the bottom of second chamber 5, i.e. at the edge of the membrane and container 19. In the embodiment shown, the supply channel has an angular cross-section. Alternatively, the supply channel may have a rounded, particularly a circular cross-section.

FIG. 9 shows a cross-sectional view of another embodiment of the device shown in FIG. 8. The device 1 has a first inlet 2 for supplying a dispersed phase, which opens into first chamber 4 having a rounded cross-section. In the embodiment shown, first chamber 4 has the shape of a spherical dome. A membrane 7 separates first chamber 4 from second chamber 5. In contrast to the embodiment shown in FIG. 2, the membrane is inclined with respect to the central longitudinal axis 15 of the device 1. The acute angle β in a cross sectional view along the central longitudinal axis between the central longitudinal axis and the second side of the membrane is between 45° and 89°, preferably between 70° and 88°, more preferably between 78° and 87°. The device 1 comprises additionally gas outlet 36. The gas outlet and the membrane are arranged such that gas within the first chamber is during supplying the dispersed phase to the first chamber, in particular during the first filling, directed towards the gas outlet and removed from first chamber 4 via the gas outlet 36. As can be seen, gas outlet 36 is arranged at the top edge of first chamber 4, which is formed by the membrane 7 and the chamber wall, which is part of the base 14. Before the initial filling of first chamber 4 with the dispersed phase, gas, particularly air, is present in the first chamber. Upon filling of first chamber 4 with the dispersed phase, air is pushed out of gas outlet 36. Due to the arrangement of membrane 7 and gas outlet 36, essentially all gas can be removed from first chamber 4. As remaining gas, in particular gas bubbles have detrimental effects on pressure distribution, size and particle distribution becomes more uniform.

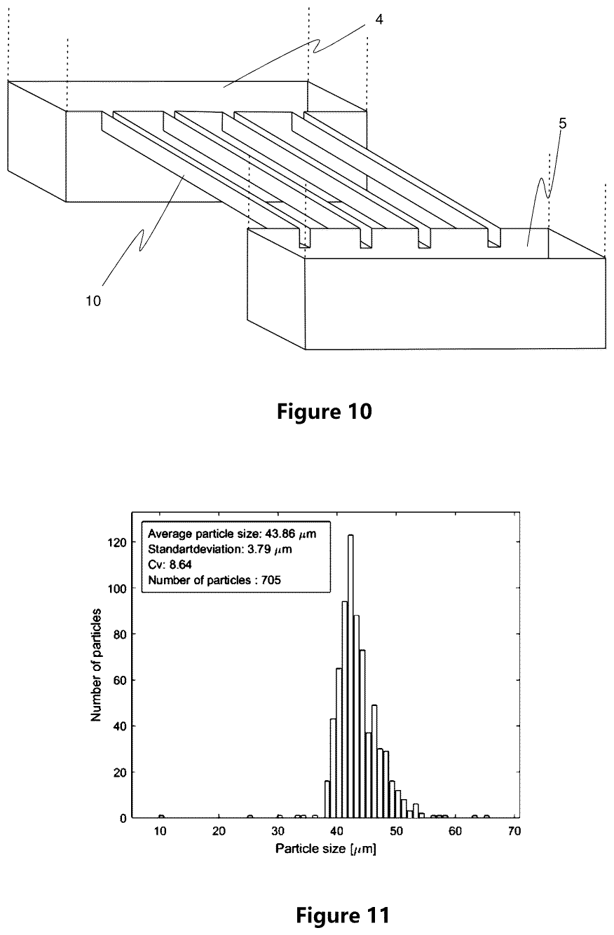

FIG. 10 shows a sectional view of another device which can be used in the method according to the present disclosure (cf. FIG. 1). The device comprises first chamber 4 being in fluidic connection via channels 10 with second chamber 5. Thus, the in the first chamber can be provided a dispersed phase as described above, which is then guided via channels 10 from the first chamber into the second chamber 5, which contains a continuous phase, the continuous phase comprising oil and at least one first surfactant.

FIG. 11 shows the size distribution of an assembly of PLGA capsules according to an embodiment of the present disclosure. As can be seen, the average particle size of 705 particles of the assembly is 43.86 μm with a standard deviation of 3.79 μm and a coefficient of variation of 8.64%.

Although the disclosed subject matter has been described in detail for the purpose of illustration based on what is currently considered to be the most practical and preferred embodiments, it is to be understood that such detail is solely for that purpose and that the disclosed subject matter is not limited to the disclosed embodiments, but, on the contrary, is intended to cover modifications and equivalent arrangements that are within the spirit and scope of the appended claims. For example, it is to be understood that the presently disclosed subject matter contemplates that, to the extent possible, one or more features of any embodiment can be combined with one or more features of any other embodiment.

1. A method for generating capsules, the method comprising the steps:
a. in a first chamber a dispersed phase, the dispersed phase comprising a solution comprising a first solvent and a matrix-forming agent, wherein the matrix-forming agent is a solid in its pure state and wherein the first solvent and the matrix-forming agent are configured such that the matrix-forming agent is soluble in the first solvent;
b. providing in a second chamber a continuous phase, the continuous phase comprising a second solvent;
wherein the first chamber and the second chamber are fluidic connected by one or more channels; and wherein the method further comprises:
c. guiding the dispersed phase from the first chamber through the one or more channels into the second chamber to form an emulsion or a dispersion comprising a plurality of droplets of the dispersed phase, in the continuous phase;
d. removing the first solvent from the droplets of the dispersed phase and solidifying the matrix-forming agent to form a capsule.

2. The method according to claim 1, wherein the dispersed phase in step a. further comprises at least one first compound of interest.

3. The method according to claim 1, wherein step a. comprises dissolving the matrix-forming agent in the first solvent to provide the dispersed phase.

4. The method according to claim 1, wherein step d. comprises the extraction of the first solvent from the droplets of the dispersed phase into the continuous phase.

5. The method according to claim 4, wherein the first solvent of the dispersed phase has a solubility of 0.1 wt. % to 40 wt. % at 25° C. and 1 atm., in the second solvent of the continuous phase.

6. The method according to claim 4, wherein a third solvent is added to the emulsion or dispersion formed in step c., wherein the third solvent has a higher solubility or a lower solubility for the first solvent of the dispersed phase as compared to the solubility of a second solvent of the continuous phase for the first solvent.

7. The method according to claim 4, wherein the extraction is performed in an extraction column.

8. The method according to claim 1, wherein the dispersed phase in step a. is an emulsion of the first solvent and a fourth solvent, wherein the emulsion comprises at least one second surfactant.

9. The method according to claim 1, wherein the matrix-forming agent is a polymer.

10. The method according to claim 1, wherein step d. is at least partially performed under continuous flow of the emulsion or dispersion formed in step c.

11. The method according to claim 1, wherein prior to step d. the emulsion or dispersion formed in step c. is removed from the second chamber.

12. The method according claim 1, wherein a pressure of 1.01 bar to 2.0 bar is applied to the first chamber and/or wherein a pressure of 1.02 bar to 1.2 bar is applied to the second chamber.

13. The method according to claim 12, wherein the pressure applied to the first chamber is smaller than the pressure applied to the second chamber.

14. The method according to claim 1, wherein step d. is performed for 0.01 min to 48 h min.

15. The method according to claim 1, wherein after step d. the formed capsules are isolated, dried, cured and/or preserved.

16. An assembly of capsules comprising a plurality of capsules produced according to the method according to claim 1.

17. The assembly of capsules according to claim 16, wherein the capsules have an equal size distribution with a coefficient of variation of 10% or less.

18. A device for generating capsules comprising
a. a first inlet for supplying a dispersed phase, opening into a first chamber;
b. a second inlet for supplying a continuous phase, opening into a second chamber, wherein the first chamber and the second chamber are fluidic connected by one or more channels, wherein each channel comprises a channel inlet opening into the first chamber and a channel outlet opening into the second chamber;
c. a dispersion outlet for collecting an emulsion or dispersion of the dispersed phase in the continuous phase; and
d. an extraction column being fluidic connected to the dispersion outlet;
wherein the first chamber is configured such that a flow rate of the dispersed phase through each individual channels is essentially uniform.

19. The device according to claim 18, wherein the first chamber has a rounded cross-section.

20. The device according to claim 19, wherein the first chamber has a hemispherical shape and the first inlet is arranged adjacent to a pole of the hemisphere-shaped first chamber.

21. The device according to claim 18, wherein the one or more channels are comprised in a membrane separating the first chamber and the second chamber comprising a first side facing the first chamber and a second side facing the second chamber, wherein the one or more channels extend from the first side to the second side of the membrane providing a fluidic connection between the first chamber and the second chamber, wherein each channel inlet is arranged on the first side of the membrane and each channel outlet is arranged on the second side of the membrane.
